(19) United States
(12) Patent Application Publication
(10) Pub. No.: US 2021/0220550 A1
(43) Pub. Date: Jul. 22, 2021

(54) Treatment Mobility Apparatus

(71) Applicants: Lynette Tiffanie KRASNER, Justin, TX (US); Joseph Daniel KRASNER, Justin, TX (US)

(72) Inventors: Lynette Tiffanie KRASNER, Justin, TX (US); Joseph Daniel KRASNER, Justin, TX (US)

(21) Appl. No.: 16/684,156

(22) Filed: Nov. 14, 2019

(51) Int. Cl. A61M 5/14

(52) U.S. Cl. CPC A61M 5/1415; A61M 2209/082

(57) ABSTRACT

An improved intravenous pole having greater flexibility, stability, and mobility. The preferred embodiment is a treatment mobility apparatus. The device is an improved advancement in existing IV pole technology. Applicants' device has a unique upper base member that allows for greater mobility for both the patient and the pole itself. The upper base member has a support rod and divots to prevent the pole from tilting or falling, which increases the stability of the device. Further, at the top of the rod, there is an opening where multiple hook devices can be inserted and interchanged allowing for flexible use of the device.

### BACKGROUND OF THE INVENTION

### Field of the Invention

The present invention generally relates to devices used to support intravenous (IV) fluid bags and tubes and feeding tubes and, more particularly, to a device that supports a variety of medical equipment with greater flexibility, stability, and mobility.

### Background of the Invention

When a person is attached to an IV pole every step is a risk. IV poles are attributable to nine percent of fall-related injuries in hospitals. The risk of falling with an IV pole is one of the top five highest variables in predicting a pediatric fall. Three percent of hospital patients suffer a fall while in hospital care. Falls often lengthen hospital stays and result in higher costs of care. The risk of falls in conjunction with IV poles is so widely known that most hospitals have specific policies regarding fall prevention and IV pole usage.

IV poles are relatively rudimentary medical devices that have existed for at least a century. Despite the everyday use of IV poles in doctors' offices, clinics, hospitals, and homes, the standard design of IV poles has not changed over time, notwithstanding the need for an improved design. The purpose of an IV pole is to give patients vital fluids in a comfortable manner. However, because of the size and inflexibility of standard IV poles, the stands do not always make the patient more comfortable and can contribute to patient injuries. Use of an IV pole is one of the most common variables in hospital falls for both elderly and pediatric patients.

Free-standing IV poles are usually tall, metal, mobile devices. Most IV poles are very similar with their differences attributed to the base of the stand. While the stands are easy to move over smooth and even surfaces, they are prone to falling over, which leads to patient injuries. IV poles are unstable because the stands have small bases relative to the size of the tall pole and are generally made of lightweight material. IV poles are particularly impractical for use with pediatric patients.

The base is the supporting structure for IV poles. The designs of standard IV pole bases vary but have common characteristics. Standard IV poles have “legs” which extend outward and downward from the pole with wheels attached at the end. The most common variation in IV pole design is in the number and arrangement of the legs and size of the base itself. Standard IV poles are adjustable and range from 40 to 80 inches.

The free-standing IV pole is most commonly used in hospitals. While IV poles are generally used in hospitals, people with feeding tubes and IV needs use IV poles at home in their everyday life. Hospitals are constructed with medical devices in mind, but homes typically are not. As a result, use of a standard IV pole in the home presents risk to the user that may not be encountered in the hospital. For example, flooring in homes may possess seams or other obstructions which may impede the movement of an IV pole, which may harm the user by causing a fall or tangling of attached tubing.

The standard IV pole has one axis of rotation facilitated by wheels or other rolling members attached to the base of the pole. The use of wheels to allow rotation creates significant risk to the user. The wheels may be obstructed by objects on the ground or simply get stuck. As a result, rather than following smoothly with the user, tubes may wrap around the pole as the user moves. The tangled tubes may cause the IV pole to get pulled over, or for the patient to trip and fall.

The fall of the patient or the IV pole can cause the sudden removal of a needle used for IV treatment or a feeding tube. Immediate removal of an IV needle (“needle dislodgement”) may cause severe bleeding and damage to blood vessels. The sudden removal of a feeding tube may cause a variety of severe symptoms, such as separation of the stomach from the abdominal wall and infection. Surgery may be required to replace the removed feeding tube.

In addition to physical injuries an IV pole may cause, the use of an IV pole can have negative psychological, developmental, physical, and social impacts on the user of the IV pole and that person's caretaker. Young children develop psychologically, developmentally, physically, and socially through exploratory play and testing their limits. In addition, play is vital for the social development of young children. However, the standard IV pole presents an obstacle for children to play and interact with their peers. As mentioned above, the standard IV pole presents serious medical risks to patients, particularly those who are physically active. Caregivers often seek to limit the risk to patients by limiting risky activities, especially for young children.

The benefits and risks that IV poles present also affect caregivers. In fact, IV poles produce greater benefits to caregivers, mainly nurses, than they do for patients. However, the physical risks and the difficulty of maneuvering standard IV poles present problems of anxiety and stress that affect the caregiver, especially when the caregiver is a family member. For caregivers taking care of children, the current designs in IV poles force parents and nurses caring for children to be on high alert all the time. When a child must use an IV pole, the caregiver's attention becomes focused on chasing after the IV pole to prevent a child from injuring himself or herself and not on other aspects of the child's care. The constant monitoring of a child not only is exhausting to a caregiver but can affect the caregiver's relationships with other family members.

Applicants' device provides for quick disassembly so that the device is easily transportable. This feature is especially helpful to caregivers who are parents of children. Parents with young children are frequently on the go, particularly those with chronically ill children. Parents with chronically ill children often take their child to several medical appointments per month. The ability to quickly disassemble and reassemble the IV pole alleviates significant stress and inconvenience. Traveling with children is extraordinarily difficult when children are healthy, and that difficulty is compounded when a family has a child whose mobility is impaired. For some children, like those in wheelchairs, decreased mobility is a circumstance that the family and child will have to live with. For children with IV poles, that decreased mobility does not have to be a lifelong problem. Applicants' device eases mobility issues and allows a child to have more independence and safety while traveling.

### SUMMARY OF THE INVENTION

A preferred embodiment of the present invention will provide a novel and unobvious IV pole which contains a mechanism for locking an IV tube, feeding tube, or other treatment tube in place with a divot and flap on the upper base member. This is also known as the tubing lock and securing door located on the upper base member. The tubing lock and securing door on the upper base member prevent the tubing from being wrapped around the pole. While in use, the tubing sits in the divot and the tubing is held in place by a flap, latch, or securing door. In addition, the tubing lock and securing door on the upper base member reduce the pulling force applied by the patient on the mounted devices. Such mounted devices, like a feeding pump, could have the feeding bag (or other tubing) forcibly removed from the feeding pump (or other medical devices), and could cause damage to the feeding pump (or other medical devices).

It is another preferred embodiment of the present invention to provide a novel and unobvious IV pole which permits a plurality of different mountable hooks to allow for a variety of uses by the patient and caregiver.

It is another preferred embodiment of the present invention to provide a novel and unobvious IV pole which permits quick disassembly so the device can be transported.

It is another preferred embodiment of the present invention to provide a novel and unobvious IV pole which has a support pole that is fastened to an upper base member that rotates on a Lazy-Susan-like component that has been mounted to the base. The free rotation of the upper base member from the base reduces tension on the tubing, patient, and medical devices. The free rotation of the upper base member allows for greater freedom of mobility and security of the patient by ensuring that tubes remain oriented toward the patient. Twisting of the tubing around the pole could cause injury to the patient by either removing the tubing from the mounted device or causing the tubing to be forcibly removed from the patient's body port, such as a feeding tube or IV tube.

It is another preferred embodiment of the present invention to provide a novel and unobvious IV pole which has a circular or substantially circular handle attached to the base to provide ease of movement. The handle is expected to be used by the user and by therapists and caregivers who assist the user. The handle may be used as a support for the person using the IV pole or for caregivers to assist them in transporting the IV pole. In a preferred embodiment of the invention, the circular or substantially circular handle has a bumper edge guard that is especially useful in reducing damage to walls and furniture in an IV pole user's home. Standard IV poles can get caught on furniture, which compromises the patient's safety, because the patient may not realize the IV pole is stuck and continue to move about. The immediate stop of the device's movement could cause the patient's medical ports to dislodge from the patient's body; such removal can be painful and abrupt and cause serious injury that requires emergency medical assistance. The placement of this handle at the base of the pole allows for the pole to be safely pushed and pulled at the bottom of the apparatus. This feature is particularly useful for children, who are typically shorter in stature, which makes it difficult to maneuver the pole. The standard IV pole lacks a handle near the base, which makes it difficult for shorter and weaker patients to safely maneuver the pole.

It is another preferred embodiment of the present invention to provide a novel and unobvious IV pole where the circular or substantially circular handle also functions as a finger guard. The finger guard protects small objects, including fingers of the user, from being pinched by the Lazy-Susan-like component or otherwise rotating base and upper bank member.

It is another preferred embodiment of the present invention to provide for the use of different mountable hooks to be used by the patient and caregiver for a variety of reasons.

The design of the Applicants' IV pole reduces the physical concerns IV poles present regarding injuries and mental and emotional concerns that burden caregivers. Applicants' design includes features that reduce injuries and increase the stability, mobility, and flexibility of the IV poles.

### BRIEF DESCRIPTION OF THE DRAWINGS

FIG. 1 is a perspective view of the preferred embodiment of the invention with the view angled down toward the top of upper base member 4.

FIG. 2 is a perspective view of the preferred embodiment of the invention with the view angled up toward the bottom of base 6.

FIG. 3 is an exploded perspective view of the preferred embodiment of the invention.

FIG. 4 is a cross section view of the interaction between primary pole 2 and upper base member 4, base 6, and cap 8 showing said primary pole detached from the base of the apparatus.

FIG. 5 is a cross section view of the interaction between primary pole 2 and upper base member 4, base 6, locking pins 12, grooves 14, and cap 8 showing said primary pole attached to the base of the apparatus.

FIG. 6 is a top plan view of the invention demonstrating the rotation of upper base member 4 around the central axis of the apparatus.

FIG. 7 is a perspective view of the top ends of primary pole 2 and sliding pole 18 and how they may lock with hook 22.

FIG. 8 is a perspective view of the top ends of primary pole 2 and sliding pole 18 and how they may lock with hook 24.

FIG. 9 is a perspective view of the top ends of primary pole 2 and sliding pole 18 and how they may lock with hook 26.

### DETAILED DESCRIPTION OF THE PREFERRED EMBODIMENT

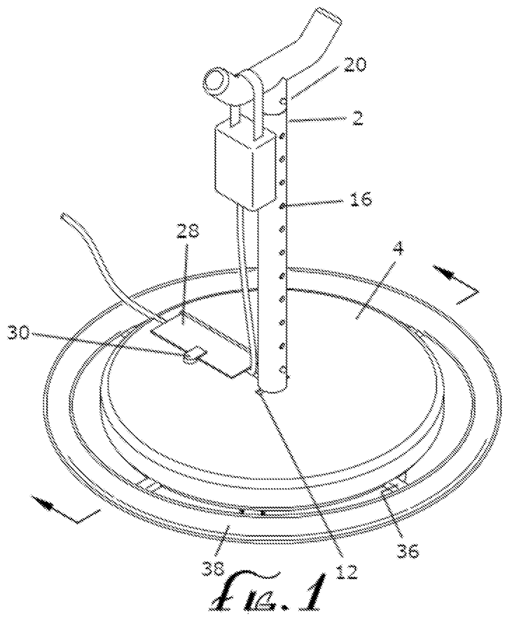

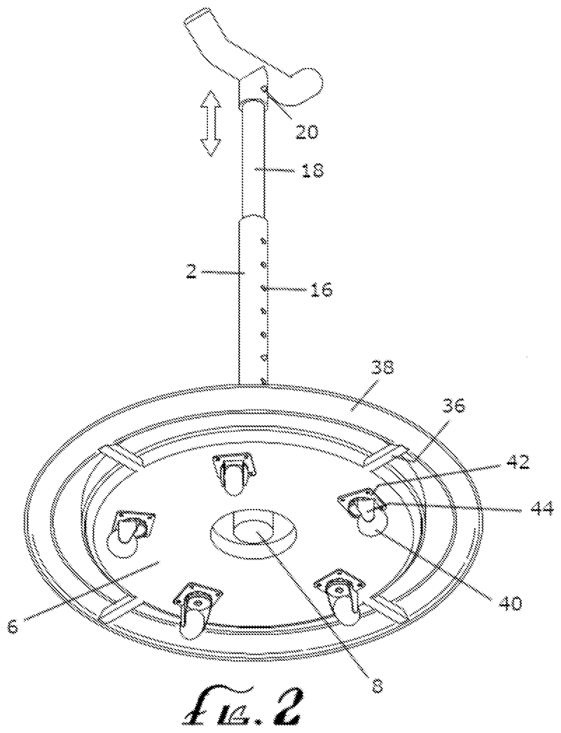

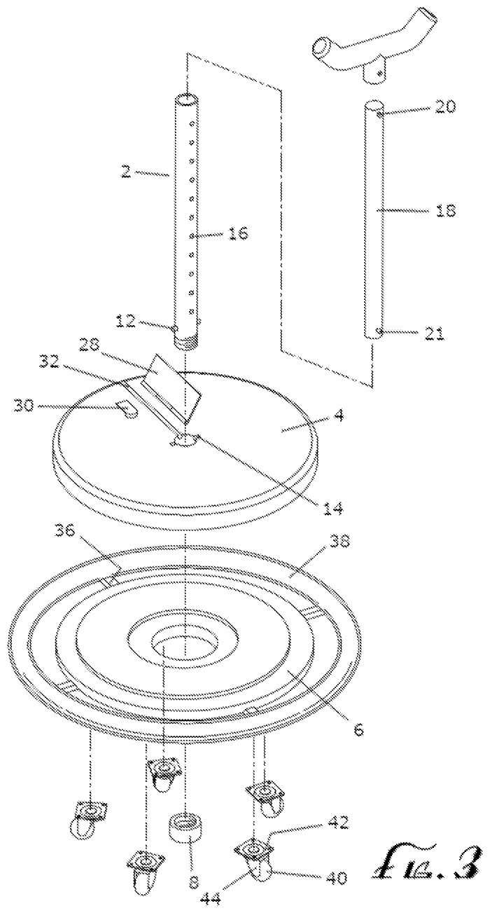

Referring to FIGS. 1, 2, 3, 4, and 5, the IV stand of Applicant's invention is identified by the reference numeral 10.

Applicant's approach to providing an IV stand has a revolutionary aspect providing two modes of rotation, with one mode around the wheels connected to the base of the stand and the other mode around a freely rotating upper base member. This revolutionary aspect allows for greater safety and stability for the user. In addition, Applicant's invention allows for quick disassembly and portability.

An embodiment of the present inventor's IV stand comprises a pole structure fastened to a base structure.

The pole structure comprises a hollow primary pole 2 which is fastened to the base structure through the male screw threading located at the bottom of primary pole 2. Primary pole 2 passes through a similarly sized opening in the upper base member 4 and larger opening in the base 6. Primary pole 2 screws into the cap 8 which sits inside of overlapping openings in upper base member 4 and base 6 and has female screw threading. Cap 8 is smaller than the opening in base 6 and larger than the opening in upper base member 4.

Primary pole 2 contains a pair of spring-loaded locking pins 12. Locking pins 12 consist of depressible spring-loaded pins extending perpendicularly from primary pole 2 at a position in which the pegs will sit in corresponding grooves 14 in upper base member 4 when primary pole 2 is fastened to base 6 by way of cap 8.

Primary pole 2 contains a plurality of locking holes 16. Primary pole 2 has two rows of locking holes 16 which are oriented opposite of one another. The first row and second row of locking holes are oriented lengthwise along primary pole 2 and are generally equidistant from one another.

The pole structure also comprises a sliding pole 18. Sliding pole 18 is sized such that it can be inserted into primary pole 2. Sliding pole 18 is also longer than primary pole 2. Sliding pole 18 also contains two pairs of locking pegs: a first set of locking pegs 20 and a second set of locking pegs 21. The first set of locking pegs 20 is positioned at the upper end of the sliding pole 18 and the second set of locking pegs 21 is positioned at the lower end of the sliding pole 18. Locking pegs 21 correspond with locking holes 16 in primary pole 2 such that they lock together and hold sliding pole 18 in place when sliding pole 18 is inserted into primary pole 2. The length of sliding pole 18 and the positions of the first set of locking pegs 20 and the second set of locking pegs 21 are such that the first set of locking pegs 20 will not enter primary pole 2 and remain exposed when sliding pole 18 is fully inserted into primary pole 2. The first set of locking pegs 20 may be used to attach a variety of hooks, exemplified by 22, 24, and 26, to sliding pole 18.

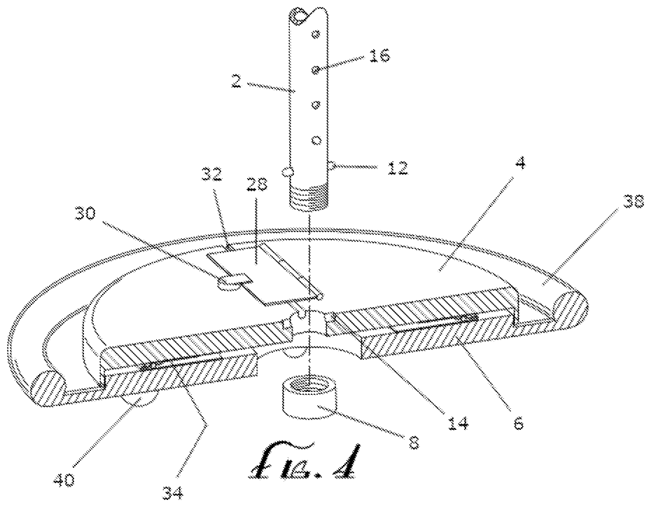

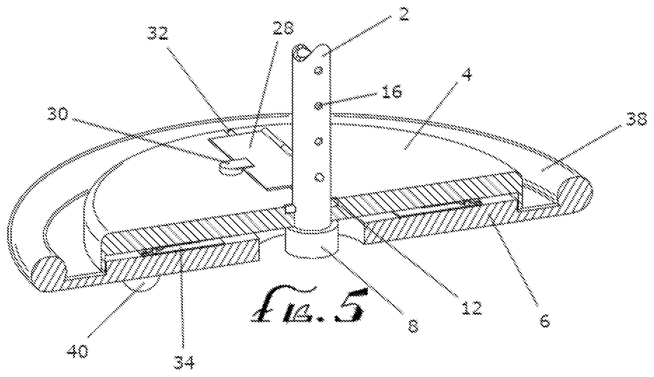

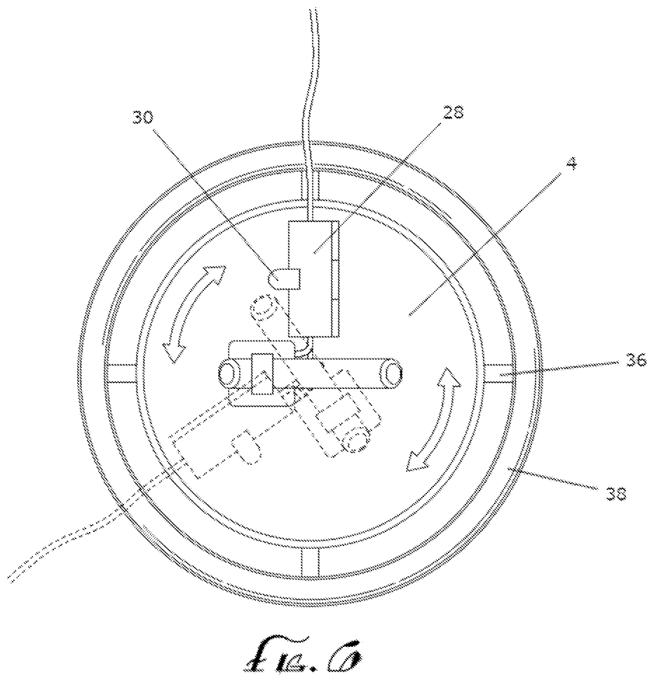

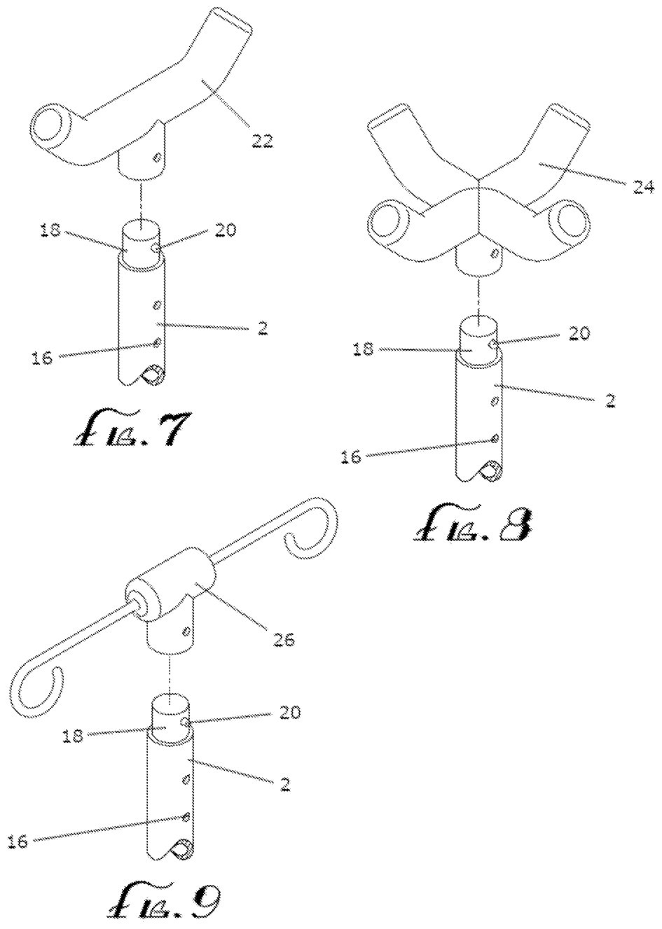

With further reference to FIGS. 6, 4, and 5, these figures illustrate the interlocking functions of upper base member 4, base 6, primary pole 2, and cap 8.

Attached to upper base member 4 are flap 28 and latch 30. Latch 30 pivots and may be used to hold flap 28 in place. IV bag and tube in groove 32 are merely illustrative and show where the IV bag and tube could be located and how the IV tube may be secured by flap 28 and said latch 30 into groove 32 in upper base member 4.

Upper base member 4 secures into base 6. Base 6 consists of a track structure referenced as 34, a first, second, third, and fourth bridge component referenced as 36, and a handle 38. Bridge components 36 connect base 6 to handle 38. A plurality of pivotally attached rolling members 40 are attached to wheel bases 42 though axels 44. Wheel bases 42 are attached to base 6 in a radial pattern. A locking mechanism (not shown), including a removable pin, clamping collar, cam, drum, or caliper, prevents independent rotation of said upper base member 4 and said lower base member 6 about their respective axial centers.

1. An improved IV pole comprising:
A primary pole having an upper pole end and a bottom pole end;
An upper base member;
A lower base member;
An interchangeable product support unit configured for reversible engagement with said upper pole end of said primary pole;
Said upper base member and said lower base member being rotatably engaged, whereby said upper base member and said lower base member may rotate about their axial center independently of each other;
Said upper base member having a pole engagement opening configured for reversible engagement with said bottom pole end of said primary pole;
Said lower base member having a plurality of rolling members attached to a bottom surface thereof, opposite said lower base member's interface with said upper base member.

2. The invention of claim 1 wherein said pole engagement opening has associated therewith first locking means for reversibly locking said primary pole in an interfaced engagement with said upper base member.

3. The invention of claim 1 wherein said interchangeable product support unit includes second locking means for reversibly locking said interchangeable product support unit in an interfaced engagement with said primary pole.

4. The invention of claim 1 wherein said upper base member includes a divot and flap with third locking means for holding said flap in place over said divot.

5. The invention of claim 1 wherein said interchangeable product support unit includes fourth locking means for reversibly locking said interchangeable product support unit with various products.

6. The invention of claim 1 wherein said lower base member includes a substantially circular handle member.

7. The invention of claim 1 wherein said upper base member and said lower base member are rotatably engaged by a sliding track system.

8. The invention of claim 1 further comprising locking means for preventing independent rotation of said upper base member and said lower base member about their respective axial centers.

9. The invention of claim 1 wherein said upper base member and said lower base member are rotatably engaged by a plurality of second rolling members.

10. The invention of claim 1 wherein said upper base member and said lower base member are rotatably engaged by a radial ball bearing rotation mechanism.

11. The invention of claim 1 wherein said bottom pole end of said primary pole and said upper base member are engaged with a screw thread securing system.
